(19) United States
(12) Patent Application Publication
(10) Pub. No.: US 2013/0109036 A1
(43) Pub. Date: May 2, 2013

(54) PROTEIN SUBSTRATE AND ITS MANUFACTURING METHOD

(71) Applicants: YI-LING YE, YUNLIN COUNTY (TW); LI-SHIAN SHI, YUNLIN COUNTY (TW); LIN-YI WU, YUNLIN COUNTY (TW); I-EN LIN, YUNLIN COUNTY (TW); JENG-HAUR HORNG, YUNLIN COUNTY (TW); L. JAMES LEE, YUNLIN COUNTY (TW)

(72) Inventors: YI-LING YE, YUNLIN COUNTY (TW); LI-SHIAN SHI, YUNLIN COUNTY (TW); LIN-YI WU, YUNLIN COUNTY (TW); I-EN LIN, YUNLIN COUNTY (TW); JENG-HAUR HORNG, YUNLIN COUNTY (TW); L. JAMES LEE, YUNLIN COUNTY (TW)

(73) Assignee: NATIONAL FORMOSA UNIVERSITY, YUNLIN COUNTY (TW)

(21) Appl. No.: 13/284,812

(22) Filed: Oct. 28, 2011

(51) Int. Cl. G01N 33/53; C12P 21/00

(52) U.S. Cl. 435/7.95; 435/68.1

(57) ABSTRACT

A protein substrate includes a base having micro-passages and reaction grooves, a polyethyleneimine (PEI) fixed on the base and a specific protein fixed on the PEI. The base is modified by plasma stripper to make the PEI bonded on the base. Mixed with tyrosinase, the specific protein can stably stick on the base due to the tyrosinase bonded with the PEI. By antigen-antibody bonding specificity, the invention can quickly detect antibody in a sample able to link with the specific protein. The specific antibody can be added with targeted biotin secondary antibody, targeted avidin-peroxidase, and color producing substrate able to react with the targeted avidin-peroxidase, such as tetramethylbenzidine or 3-(4-hydroxy)phenyl propionic acid (HPPA), so as to measure a specific antibody amount.

### BACKGROUND OF THE INVENTION

1. Field of the Invention

This invention relates to a protein substrate and its manufacturing method, particularly to one fixing a specific protein on a base to detect a specific antibody by indirect enzyme-linked immunosorbent assay, based on antigen-antibody bonding specificity.

2. Description of the Prior Art

Commonly, Enzyme-linked immunosorbent assay (ELISA) has been employed to qualitatively detect and quantitatively measure antigens or antibodies with a variety of enzymes since it was primarily disclosed by Engvall and Perlman in 1971, with a great sensitivity and specificity for the examination of antigens and antibodies. The theory of ELISA is based on a linking specificity between antigens and antibodies, with a specific antigen to identify a specific antibody or with a specific antibody to catch a specific antigen. However, as immunosorbent of the antigen-antibody is colorless, an enzyme is linked to an antibody Fc fragment to have a coloring reaction with a color producing substrate, then qualitative and quantitative analysis by an instrument. The ELISA is categorized into three types: Indirect ELISA, Sandwich ELISA and Competitive ELISA.

The Indirect ELISA is utilized to detect if a sample possesses antibodies or not. First, a blood serum or a sample containing a primary antibody (Ab1) is put into a particular antigen-coated microtiter well to react with the specific antigen, with the free primary antibodies washed off. Next, an enzyme-conjugated secondary anti-isotype antibody (Ab2) is added to react with the primary antibody, with the Ab2 unlinked to the Ab1 washed off. Finally, an enzyme substrate is added. Colored product can be quantitatively measured by a particular spectrophotometric plate reader. Any antigen specific antibodies can be detected by the indirect ELISA. In clinical application, Indirect ELISA is common to detect if a serum has antibodies able to combat with a specific bacterium or a specific antigen, so as to find out if a patient is infected by any infectious organisms. For instance, it can detect if a person has antibodies against Hepatitis B virus (HBV) and Human immunodeficiency virus (HIV).

The indirect ELISA has advantages as follows.

1. It can detect particular antigens and antibodies qualitatively and quantitatively, with a high sensitivity and with a magnifying effect.

2. As it has been widely used, a great variety of reagents, consumptive materials and instruments have been provided to satisfactorily meet diverse experimental purposes needing a convenient combination of them, such as clinical detection, biochemical experiments, environmental detection and food examination.

3. The microtiter well can be made of various materials. Polystyrene is usually selected for optical instruments as it is able to be preserved for a long term withstanding a wide range of temperature change, even possible to be stored in −80° C. Moreover, it can be easily manufactured by molds, with a low cost and a good plasticity.

However, the Indirect ELISA also has disadvantages as follows.

1. A whole analysis process, with a 96-well microtiter used as a fixed carrier, needs to run a day and half approximately, too long for an emergency.

2. A large amount of sample (100 μL) is needed. If it is necessary to do detection for diverse cytokines simultaneously, some dear samples may run short of. Relatively, consumption of reagents is stepped up as well. Thus the whole cost is high.

3. Fluidic treatment is pretty complicated as each stage needs a clean-up to keep optical instruments from being interfered by impurities.

### SUMMARY OF THE INVENTION

The conventional Indirect ELISA has disadvantages as mentioned below. It takes approximately one day and half to detect antigen or antibody, too long for an emergency. As a reagent can only detect one antigen or one antibody owing to antigen-antibody linking specificity, a great number of reagents and samples (100 μl) are needed if a variety of antigens or antibodies have to be detected, not only posing a soaring detection cost if a reagent is expensive, but also possible to be short of samples. Moreover, in order to assure linking specificity, a lot of complicated fluidic treatments must be done, apt to cause operational errors.

The object of this invention is to offer a manufacturing method of a protein substrate that can quickly detect a specific antibody, cutting out some complicated processes and lowering the amount of consumptive materials.

Another object of this invention is to offer a protein substrate fixed with a specific protein to quickly detect a specific antibody.

The manufacturing method of the protein substrate is to modify a base so that it can chemically, stably bond with a specific protein for detecting a specific antibody. The manufacturing method includes the following steps. First, the base is modified by a plasma stripper with a power of 300 watts to link with —OH group. Second, 10 microliters of polyethyleneimine is used to react with the —OH group of the base, with —NH₂ group exposed. Third, the specific protein expected to detect a specific antibody is mixed with tyrosinase by a ratio of 6:1 to 4:1, for example 5:1. The mixture is then added onto polyethyleneimine to make tyrosinase chemically bonded with the —NH₂ group, so enabling the specific protein stably fixed on the base.

The protein substrate is formed as a micro-fluidic disc, with a specific protein fixed on the base to detect a specific antibody. The base is provided with micro-passages and reaction grooves, with a polyethyleneimine fixed on the base, and with a specific protein fixed on the polyethyleneimine. The surface of the base is modified by a plasma stripper. The amount of the polyethyleneimine used is 10 microliters. The specific protein is mixed with tyrosinase by a ratio of 6:1 to 4:1, for example 5:1. 10 microliters of the mixture is used. We called it as CD (compact disc)-ELISA system.

### BRIEF DESCRIPTION OF DRAWINGS

This invention is better understood by referring to the accompanying drawings, wherein:

FIG. 1 is a flow chart of a preferred embodiment of manufacturing a protein substrate in the present invention;

FIG. 2 is a flow chart of illustrating a theory of how to modify an acrylic base in the preferred embodiment of a protein substrate in the present invention;

FIG. 3 is an experimental flow chart of detecting anti-ovalbumin antibody (anti-OVA Ab) by the preferred embodiment of an ovalbumin protein substrate in the present invention;

FIG. 4 is a whole experimental picture by the preferred embodiment of a protein substrate in the present invention;

FIG. 5 is a CD-ELISA signal detecting system used in the present invention;

FIG. 6 is a practical result measured from a CD of the present invention by the CD-ELISA signal detecting system;

FIG. 7 is a practical result of anti-ovalbumin antibody (anti-OVA Ab) in serum using a conventional 96-well microtiter and tested by the CD-ELISA signal detecting system; and

FIG. 8 is a practical result of serial diluted serum of ovalbumin (OVA) sensitized mice measured by the CD of the invention and the CD-ELISA signal detecting system.

### DETAILED DESCRIPTION OF THE PREFERRED EMBODIMENT

A preferred embodiment of a protein substrate in the present invention is formed as a micro-fluidic disc employed to detect a specific antibody by a specific protein fixed on the disc. The protein substrate is provided with a base built with micro-passages and reaction grooves, a polyethyleneimine (PEI) coated on the base, and a specific protein mixed with a tyrosinase (TR) for being fixed on the polyethyleneimine (PEI).

In this embodiment, ovalbumin is used as the specific protein and acrylic (PMMA) is used as the base, but not limited.

The reaction grooves of the acrylic (PMMA) have their surfaces modified by a plasma stripper (O₂ plasma) to enable the —OH group of the acrylic (PMMA) to react with the polyethyleneimine (PEI), so that the polyethyleneimine (PEI) can be coated on the acrylic (PMMA), with —NH₂ group exposed. By means of the tyrosinase (TR), the ovalbumin can chemically react with the —NH₂ of the polyethyleneimine (PEI) to fix thereon.

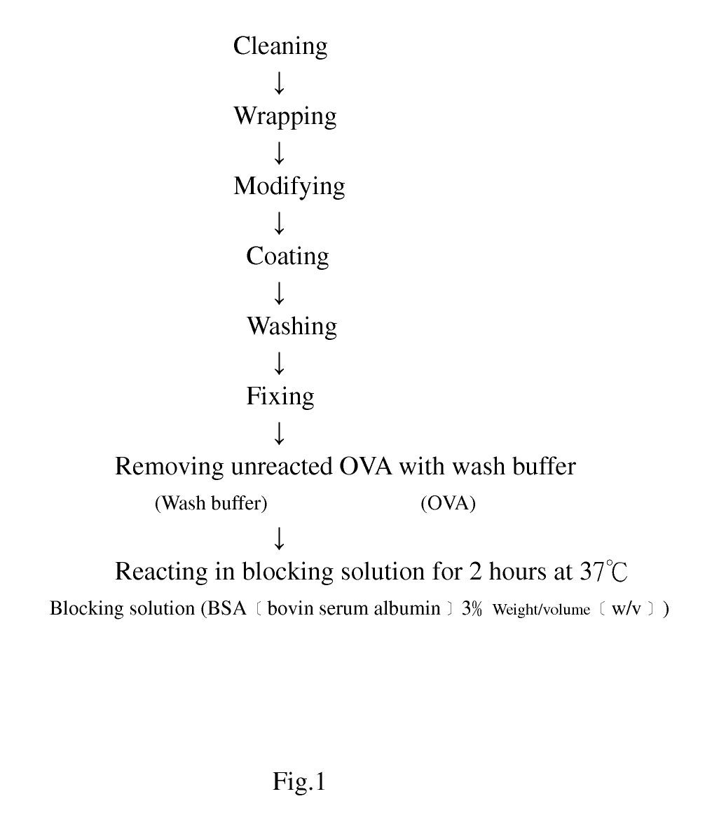

As shown in FIG. 1, the protein substrate of the invention is made by the steps described below.

1. Cleaning:

Wash the surface of the acrylic (especially the reaction grooves) with double distilled water (ddH2O) by a soft brush and dry it.

2. Wrapping:

Wrap up the surface of the acrylic with an aluminum foil completely, with the locations of the aluminum foil attached to the reaction grooves cut open.

3. Modifying:

Use a plasma stripper (300 watts for power and 0.5-2 minute, for example 1 minute for reaction time) to modify the acrylic surface of the reaction grooves to be linked with —OH group. The aluminum foil is removed afterward.

4. Coating:

Add 10 microliters (μl) of polyethyleneimine (PEI) into the acrylic surface of the reaction grooves for 0.5-2 hour, for example 1 hour to enable the polyethyleneimine (PEI) to react with the —OH group of the acrylic, with the —NH₂ group exposed.

5. Washing:

Wash the acrylic surface linked with the polyethyleneimine (PEI) with double distilled water (ddH₂O) and then dry it.

6. Fixing:

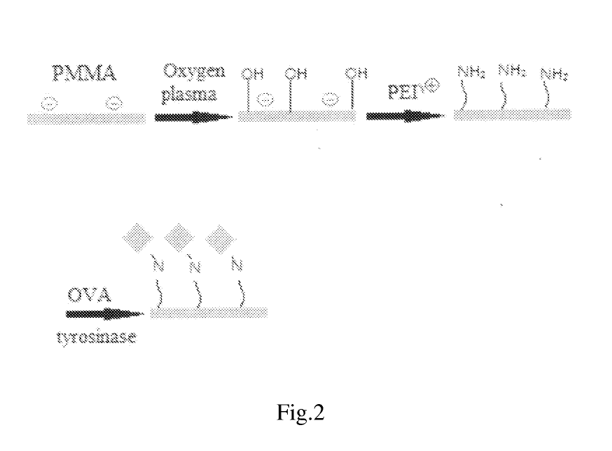

Prepare a mixture of ovalbumin and tyrosinase by a ratio of 6:1 to 4:1, for example 5:1. Add 10 microliters (μl) of the mixture onto the polyethyleneimine (PEI) of the reaction grooves for 8-10 hours' reaction at 2-6° C., for example 4° C. By means of the tyrosinase, the ovalbumin can react with the —NH₂ group of the polyethyleneimine (PEI) to fix on the reaction grooves. The theory of the experiment described above is shown in FIG. 2.

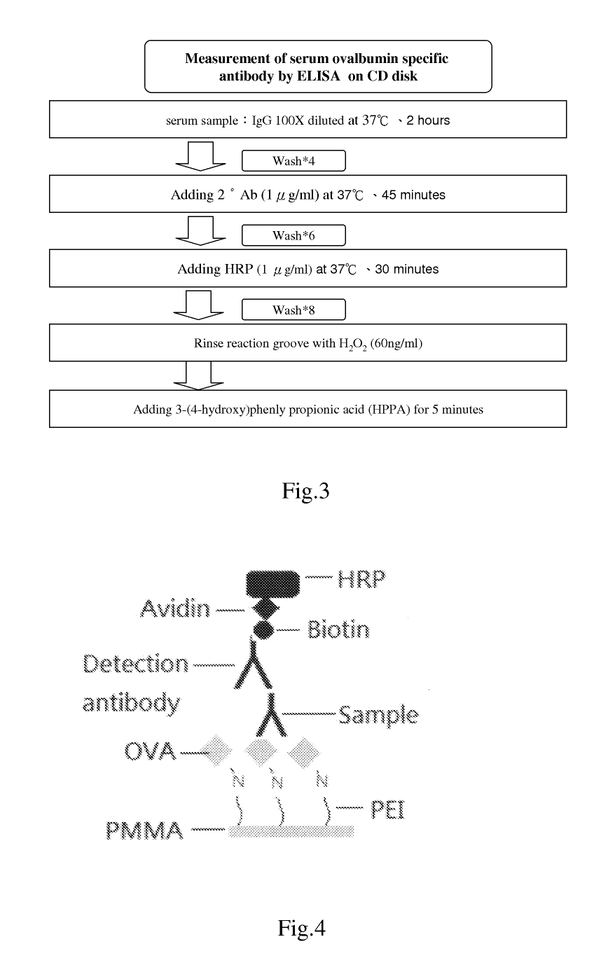

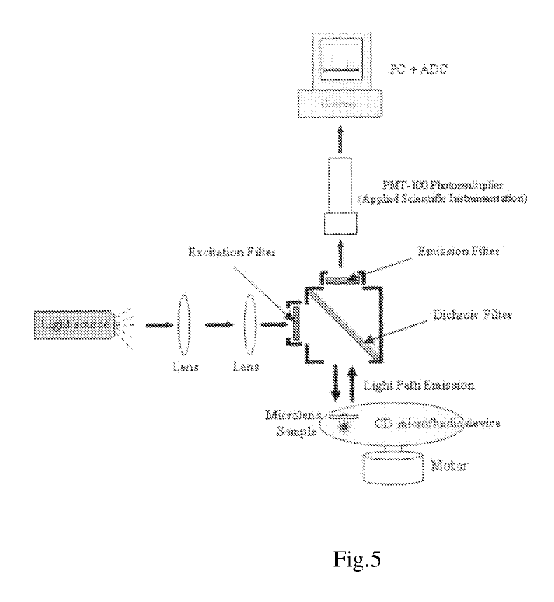

The protein substrate is finished after the ovalbumin is fixed on the reaction grooves, utilized to detect specific antibody in a blood serum able to be linked with the ovalbumin by indirect ELISA. The operation flow is shown in FIG. 3. FIG. 4 depicts a whole detecting picture by using the protein substrate manufactured in accordance with all of the steps mentioned above. FIG. 5 shows a specially designed signal detection system for detecting a sample.

The following description is to detect antibody in a sample able to link with ovalbumin by Indirect ELISA, using the protein substrate of this invention and a conventional fixed carrier respectively. a color producing substrate is added in a last step to check if the sample has antibody(anti-ovalbumin Ab) able to link with ovalbumin: if color is revealed, it represents the sample has antibody able to link with ovalbumin; if no color is revealed it represents the sample has no antibody able to link with ovalbumin. That is, with the color revealed or not, it can tell if a sample has a specific target.

1. Using the protein substrate of this invention to detect antibody in a blood serum able to link with ovalbumin by Indirect ELISA:

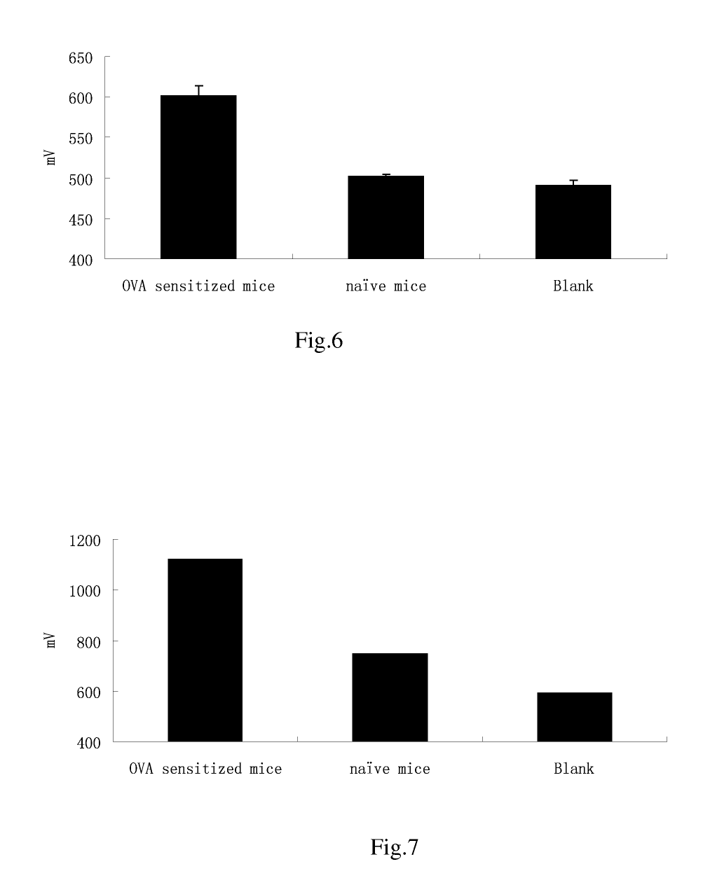

As shown in FIG. 4, on the disc fixed with ovalbumin, 20 microliters of blocking buffer is added into each reaction groove for 1-3 hours, for example 2 hours at 15-35° C., for example at room temperature (20-30° C.). Next, the disc is washed twice and the diluted blood serum is added into the reaction grooves. As IgG is the antibody to be detected, the serum is diluted with blocking buffer 70-130 times, for example 100 times. Each reaction groove is added with 20 microliters of the serum solution from different treated mice (naïve, ovalbumin sensitized mice) for 1-3 hours, for example 2 hours at 15-35° C., for example at room temperature (20-30° C.). and washed four times. Blocking buffer added only is as blank group (Blank). Each reaction groove is next added with 20 microliters of 2^{nd} Ab (1 μg/ml) for 30-60 minutes, for example 45 minutes and washed six times. Then each reaction groove is added with 20 microliters of targeted avidin-peroxidase (1 μg/ml) for 20-40 minutes, for example 30 minutes and washed eight times. And 3-(4-hydroxy)phenly propionic acid (HPPA) is added to react for 1 minute. Finally, the sample is detected by the particularly designed signal detection system shown in FIG. 5. With a light source having an emission of 320 nm wavelength, HPPA emits fluorescence with 405 nm wavelength. The detection result is shown in FIG. 6. Naïve mice are regarded as a judgment for a mice serum background value (Naïve mice). Blocking buffer is used as a negative control (Blank), representing no antibody detected to be linked with ovalbumin.

2. Using a conventional fixed carrier to compare the results of CD disc we mentioned above. The specific anti-ovalbumin antibody titer could also be measured by the same blood serum from naïve and sensitized mice by Indirect ELISA:

First, ovalbumin (10 μg/ml) is added in a 96-well microtiter for 7-9 hours, for example 8 hours at 2-6° C., for example 4° C. so as to fix on the microtiter. Next the ovalbumin unfixed to the microtiter is washed off. Each well is added with 300 μL of blocking buffer for 0.5-1.5 hours, for example 1 hour at 15-35° C., for example at room temperature (20-30° C.). Then each well is added with 100 μL of mouse serum diluted 70-130 times, for example 100 times or blocking buffer only for 0.5-1.5 hours, for example 1 hour and washed. Each well is successively added with 2^{nd} Ab (1 μg/ml) for 30-60 minutes, for example 45 minutes at 15-35° C., for example at room temperature (20-30° C.). and 100 μL of targeted avidin-peroxidase (1 μg/ml) for 20-40 minutes, for example 30 minutes at 15-35° C., for example at room temperature (20-30° C.). A color producing substrate, 3-(4-hydroxy)phenly propionic acid (HPPA), is added to react for 7-13 minutes. For example 10 minutes at 15-35° C., for example at room temperature (20-30° C.) without being exposed to light. With a light source having an emission of 320 nm wavelength, HPPA emits fluorescence with 405 nm wavelength. The signal detection system shown in FIG. 5 is used to carry out detection and FIG. 7 shows the detection results. Ovalbumin sensitized mice are regarded as a positive control, representing they have specific anti-ovalbumin Ab detected to be linked with ovalbumin. The results are similar to those found using the protein substrate of the invention. Therefore, it is demonstrated that using the protein substrate of the invention to detect a specific antibody in a sample can cut off some complicated detecting steps, lower consumption of consumptive materials, and shrink operation time to achieve a swift detection.

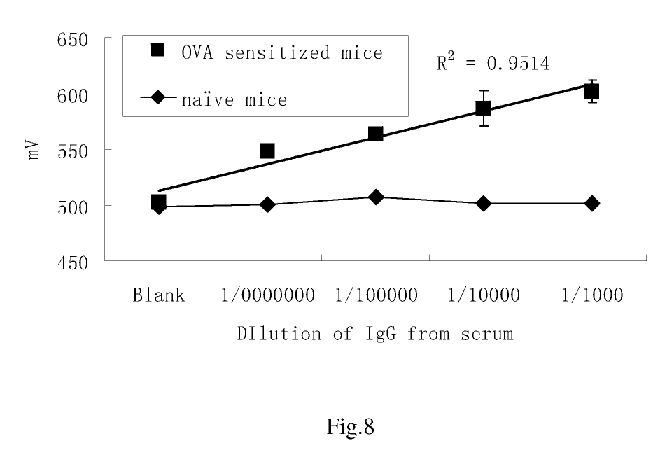

3. After a serial dilution of the serum of the ovalbumin sensitized mice (10³˜10⁶ times), a detection result is shown in FIG. 8. It is obvious that concentration-gradient relationship is found in the result, with an apparent difference with the serum of the naïve mice. It shows the stability of the detection system is good. (The result of FIG. 8 is different from what is described because there is no concentration-diluted pattern from serum of naïve mice shown in the Figure.)

While the preferred embodiment of the invention has been described above, it will be recognized and understood that various modifications may be made therein and the appended claims are intended to cover all such modifications that may fall within the spirit and scope of the invention.

What is claimed is:

1. A protein substrate formed as a micro-fluidic disc to fix a specific protein employed to detect a specific antibody, said protein substrate comprising:
a base provided with micro-passages and reaction grooves, said reaction grooves being modified by a plasma stripper to link with —OH group in conditions of 280-320 watts power and 0.5-2 minute reaction;
a polyethyleneimine added in said reaction grooves to chemically react with the —OH group on surface of said reaction grooves for 0.5-2 hours at room temperature, —NH₂ group being exposed; and
a specific protein mixed with tyrosinase by a ratio of 6:1 to 4:1 and added onto said polyethyleneimine to chemically react with said —NH₂ group of said polyethyleneimine for 8-10 hours at 2-6° C. so that said specific protein is fixed on said reaction grooves.

2. The protein substrate as claimed in claim 1, wherein said base is made of acrylic.

3. The protein substrate as claimed in claim 1, wherein said specific protein is ovalbumin.

4. The protein substrate as claimed in claim 1, wherein said reaction grooves are modified by a plasma stripper to link with —OH group in conditions of 300 watts power.

5. The protein substrate as claimed in claim 1, wherein said reaction grooves are modified by a plasma stripper to link with —OH group in conditions 1 minute reaction.

6. The protein substrate as claimed in claim 1, wherein said polyethyleneimine is added in said reaction grooves to chemically react with the —OH group on surface of said reaction grooves for 1 hour at room temperature.

7. The protein substrate as claimed in claim 1, wherein said specific protein is mixed with tyrosinase by a ratio of 5:1.

8. The protein substrate as claimed in claim 1, wherein said specific protein is mixed with tyrosinase and added onto said polyethyleneimine to chemically react with said —NH₂ group of said polyethyleneimine for 8-10 hours at 4° C.

9. A manufacturing method of a protein substrate, said manufacturing method employed to modify reaction grooves in a base to be coated with polyethyleneimine and a specific protein so as to make said protein substrate formed as a micro-fluidic disc to detect a specific antibody, said manufacturing method comprising:
a first step of using a plasma stripper to modify said reaction grooves of said base to link with —OH group in conditions of 280-320 watts power and 0.5-2 minute reaction;
a second step of adding said polyethyleneimine into said reaction grooves to chemically react with said —OH group on surface of said reaction grooves for 0.5-2 hours at room temperature, —NH₂ group being exposed; and
a third step of adding a mixture of said specific protein and tyrosinase by a ratio of 6:1 to 4:1 onto said polyethyleneimine to chemically react with said —NH₂ group for 8-10 hours at 2-6° C. so as to make said specific protein fixed on said reaction grooves of said base.

10. The manufacturing method of a protein substrate as claimed in claim 9, wherein said base is made of acrylic.

11. The manufacturing method of a protein substrate as claimed in claim 9, wherein said specific protein is ovalbumin.

12. The manufacturing method of a protein substrate as claimed in claim 9, wherein in the first step, said plasma stripper is used to modify said reaction grooves of said base to link with —OH group in conditions of 300 watts power.

13. The manufacturing method of a protein substrate as claimed in claim 9, wherein in the first step, said plasma stripper is used to modify said reaction grooves of said base to link with —OH group in conditions of 1 minute reaction.

14. The manufacturing method of a protein substrate as claimed in claim 9, wherein in the second step, said polyethyleneimine is added into said reaction grooves to chemically react with said —OH group on surface of said reaction grooves for 1 hour at room temperature.

15. The manufacturing method of a protein substrate as claimed in claim 9, wherein in the third step, said mixture of said specific protein and tyrosinase by a ratio of 5:1 is added onto said polyethyleneimine to chemically react with said —NH₂ group for 8-10 hours.

16. The manufacturing method of a protein substrate as claimed in claim 9, wherein in the third step, said mixture of said specific protein and tyrosinase is added onto said polyethyleneimine to chemically react with said —NH₂ group for 8-10 hours at 4° C.
